(19) United States
(12) Patent Application Publication
(10) Pub. No.: US 2004/0081785 A1
(43) Pub. Date: Apr. 29, 2004

(54) Infusion set and method for administrating an injection solution containing nitroglycerin

(76) Inventors: Takeshi Nizuka, Osaka-shi (JP); Hideki Mitani, Osaka-shi (JP); Mamoru Shimada, Osaka-shi (JP)

Correspondence Address: KUBOVCIK & KUBOVCIK, SUITE 710, 900 17TH STREET NW, WASHINGTON, DC, 20006

(21) Appl. No.: 10/686,622

(22) Filed: Oct. 17, 2003

Related U.S. Application Data
(62) Division of application No. 09/731,779, filed on Dec. 8, 2000.

(30) Foreign Application Priority Data
Dec. 9, 1999 (JP) 11-349790

(51) Int. Cl. F16L 1/00; A61M 25/00

(52) U.S. Cl. 428/36.9; 604/523; 604/413

(57) ABSTRACT

A medical tubing of a random copolymer containing an olefin-based monomer, such as ethylene, in an amount of 50-95 mol % and, preferably 75-95 mol %, and a styrene-based monomer, such as styrene, in an amount of 50-5 mol %, preferably 25-5 mol %. The medical tubing can be used as a conjugating tube or a connecting tube of medical equipment such as infusion circuits and blood circuits, and for administering an oil-soluble drug such as nitroglycerin. The random copolymer does not contain chlorine atoms or a plasticizer. The tubing has very small adsorption of the drugs, can withstand squeezing action by an infusion pump, has a possibility of undergoing γ-ray sterilization and has flexibility necessary for clinical use.

This application is a division of application Ser. No. 09/731,779, filed Dec. 8, 2000.

### FIELD OF THE INVENTION

The present invention relates to a medical tubing, and especially relates to a medical tubing which can constitute a part of medical equipment such as infusion circuits, blood circuits, and the like.

### BACKGROUND OF THE INVENTION

Tubes formed mainly from a soft polyvinyl chloride (PVC) containing diethylhexylphthalate (DEHP) or the like as a plasticizer have been used for a long period of time as medical tubing and, more particularly, have been used as conjunction tubes or connecting tubes of medical equipment such as infusion circuits, blood circuits, and the like. However, PVC is a homopolymer of vinyl chloride which contains a chlorine atom. Therefore, when products formed from the polymer are incinerated, there is a possibility that a harmful substance, dioxin, is generated depending on the conditions of incineration. Further, there is a possibility that DEHP contained in PVC as a plasticizer is eluted into a drug liquid charged in PVC products or into blood passed through the PVC products, and is introduced into a human body. Furthermore, there is a problem that a drug such as nitroglycerin that is required to be accurately injected is adsorbed by the tubes and a desired amount of the drug is not administered.

It is supposed that harmful dioxins are not generated when a PVC product is incinerated at a high temperature. A general small-sized incinerator is not satisfactory for such incineration because the incinerator cannot provide a high temperature. A large-sized incinerator to meet this requirement is desired but a high cost is required to install such an incinerator.

Further, DEHP contained in PVC products as a plasticizer is a low molecular weight compound and it is expected that it is easily eluted from PVC products and introduced into a human body. In particular, when a drug containing ethanol or polyethylene glycol as a dissolving agent, or blood is contained in a bag formed from PVC containing DEHP, or is passed through a tube formed from the polymer, its elution is remarkable. For this reason, PVC products not containing a plasticizer have been investigated. One example thereof is a PVC product obtained by copolymerization with an ethylene-vinyl acetate copolymer according to a specific method. However, this PVC product also contains chlorine atoms and its cost is very high. Thus, such a product can not generally be used. Further, in the case of a drug such as nitroglycerin that requires a precise injection being contained in the bag, the problem of drug adsorption has not yet been solved.

In view of the above, an infusion tube formed from a very low density polyethylene having a density of 0.89 g/cm³ or less has been proposed as a medical tubing for intravenous injection of nitroglycerin (Japanese Patent Examined Publication No. Hei 1-48775). This tube adsorbs a very small amount of nitroglycerin, and therefore an intended amount of the drug can be administered. However, there is a physical problem that although the tube has a sufficient flexibility for use as a medical tubing, the tube does not withstand squeezing by an infusion pump when an accurate flow rate is required.

The drug adsorption property of an infusion tube formed from polyvinyl chloride or polybutadiene has been discussed (M. Gerard Lee, American Journal of Hospital Pharmacy, Vol. 43, August p. 1945-1950, 1986). A tube made of polybutadiene also has a very small adsorption property with respect to a drug such as nitroglycerin, and therefore is suitable for administration of such a drug. However, since a double bond still remains in a polymer chain, storage conditions or processing conditions are restricted, or a stabilizer must be used in order to prevent a change in the physical properties of the tube during storage for a long period of time, or to prevent gelation during molding and processing of the tube. Thus, such a tube does not always satisfy safety requirements when used as a medical tube.

Further, to sterilize a tube made of 1,2-polybutadiene with γ-ray radiation, it is necessary to add a phosphorus-based antioxidant (Japanese Patent Unexamined Publication No. Hei 5-98075). Such a composition is not preferable as a medical tubing from this standpoint. Also, there is a problem that this medical tubing does not withstand squeezing by an infusion pump.

Furthermore, a resin composition comprising polybutadiene and a styrene-isoprene-styrene block copolymer is proposed as a composition suitable for medical products such as medical tubing, soft catheters, sheets and the like (Japanese Patent Unexamined Publication No. Hei 6-184360). This resin composition has very excellent flexibility, and therefore is put into practical use as an infusion tube for a pump. However, because it cannot withstand deterioration of portions squeezed by the pump, a silicone resin is coated on its surface. Moreover, because a styrene-isoprene-styrene block copolymer is unstable, there is a problem that the tube turns yellow during storage for a long period of time or in γ-ray sterilization.

Also, a drug administration tube comprising a blend of polyethylene and a styrene-based elastomer or olefin-based elastomer has been proposed (Japanese Patent Examined Publication No. Hei 2-31990). Since drug adsorption is small by this tube, the intended amount of drug can be administered. However, because an elastomer is added to polyethylene, rigidity of a molded tube decreases and there is a problem that the tube does not withstand the squeezing action of a pump.

In view of the above situation, the present inventors variously investigated medical tubing formed of a material not containing a chlorine atom or a plasticizer for the purpose of obtaining medical tubing having low drug adsorption, resistance to squeezing by a pump, a possibility of undergoing γ-ray sterilization, and a flexibility necessary for clinical usage. The inventors found that a random copolymer of an α-olefin-based monomer and a styrene-based monomer achieves the expected purpose of the present invention.

### BRIEF DESCRIPTION OF THE DRAWING

FIG. 1 is a view showing one embodiment of the use of the medical tubing of the present invention.

### SUMMARY OF THE INVENTION

The present invention relates to a medical tubing containing a random copolymer comprising an olefin-based monomer and a styrene-based monomer.

The olefin-based monomer useful in the present invention includes α-olefins. The α-olefins are α-olefin monomers having 2-20 carbon atoms, and include, specifically, ethylene, propylene, 1-butene, 1-pentene, 1-hexene, 1-heptene, 1-octene, 4-methyl-1-pentene, 2-methyl-1-propene, 3-methyl-1-pentene, 5-methyl-1-hexene, and the like.

In the present invention, the styrene-based monomer includes styrene, p-methylstyrene, t-butylstyrene, vinyl pyridine and α-methylstyrene.

The random copolymer comprising an olefin-based monomer and a styrene-based monomer useful in the present invention specifically includes a random copolymer comprising ethylene and styrene and a random copolymer comprising propylene and styrene.

In the present invention, the random copolymer comprising an olefin-based monomer and a styrene-based monomer can be a random copolymer copolymerized with another monomer such as a monomer containing a glycidyl group, amino group, dimethylamino group, hydroxyl group, carboxyl group, ester group, ether group or isocyanate group, or a maleic anhydride. The copolymerization proportion thereof is within a range that achieves the purposes of the present invention.

The copolymerization ratio of the random copolymer of the present invention is preferably one where the olefin-based monomer is 50-95 mol % and the styrene-based monomer is 50-5 mol %. More preferably, the olefin-based monomer is 75-95 mol % and the styrene-based monomer is 25-5 mol %, and most preferably, the olefin-based monomer is 80-90 mol % and the styrene-based monomer is 20-10 mol %. If the styrene-based monomer is less than 5 mol %, hardness of the tube is increased and transparency thereof is reduced. Further, if it exceeds 50 mol %, the elasticity necessary for a medical tubing cannot be obtained.

A specific example of the present invention is a medical tubing containing a random copolymer of ethylene and styrene. The random copolymer of ethylene and styrene is preferably one in which the ethylene content is 75-95 mol % and the styrene content is 25-5 mol. Further, it is particularly preferable that the ethylene content is 80-90 mol % and the styrene content is 20-10 mol %.

The ethylene-styrene random copolymer used in the present invention is a random copolymer of ethylene and styrene and optionally contains another olefin monomer.

The random copolymer herein means a copolymer in which a proportion of a block or blocks comprising two or more continuous units derived from the α-olefin based monomer or styrene based monomer is 10% or less and, preferably, 1% or less with respect to the amount of a copolymer derived from both monomers. The content of a block or blocks consisting of two or more continuous units derived from the styrene-based monomer may be determined by ¹³C-NMR.

Styrene-ethylene-butylene-styrene block copolymer (SEBS), styrene-isoprene-styrene block copolymer (SIS), styrene-butadiene-styrene block copolymer (SBS) and the like have conventionally been known as a copolymer of an olefin such as ethylene with a vinyl aromatic compound such as styrene. These copolymers have styrene block portions and olefin block portions, respectively. On the contrary, the ethylene-styrene random copolymer of the present invention is one in which ethylene and styrene are randomly copolymerized as mentioned above, and a low styrene content region does not have a block portion of styrene in the structure of the copolymer. As a result, this random copolymer substantially differs from the above-mentioned styrene-based elastomer such as SEBS.

The melt flow ratio (200° C., MFR) of the random copolymer of the present invention is 0.01-100 g/10 mins, preferably, 0.1-50 g/10 mins and, more preferably, 0.3-10 g/10 mins. The melting point is 100° C. or lower.

The random copolymer of an olefin-based monomer and a styrene-based monomer used in the present invention is synthesized by conventional methods. There are, for example, methods such as radical polymerization, ion polymerization, and the like. A high-pressure radical polymerization method is described in Japanese Patent Unexamined Publication No. Hei 1-113447. It is disclosed that the monomers are simultaneously or stepwise contacted with each other and polymerized in the presence of a radical polymerization initiator, a chain transfer agent and, if necessary, an auxiliary agent under conditions of a polymerization pressure of 500-4000 kg/cm² and a polymerization temperature of 50-400° C. in a reaction vessel.

Further, a method of producing the above-mentioned random copolymer using a metallocene catalyst is disclosed in Japanese Patent Unexamined Publication No. Hei 10-273544 and Japanese Patent No. 2,623,070. The metallocene catalyst used in these methods is a metallocene catalyst conventionally used as a single site catalyst, and a metallocene catalyst similar to such a catalyst. In particular, a catalyst comprising a metallocene compound of a transition metal (transition metal compound) and an organic aluminum oxy compound and/or an ionized ionic compound is preferably used. The above Japanese patent publications are incorporated herein by reference.

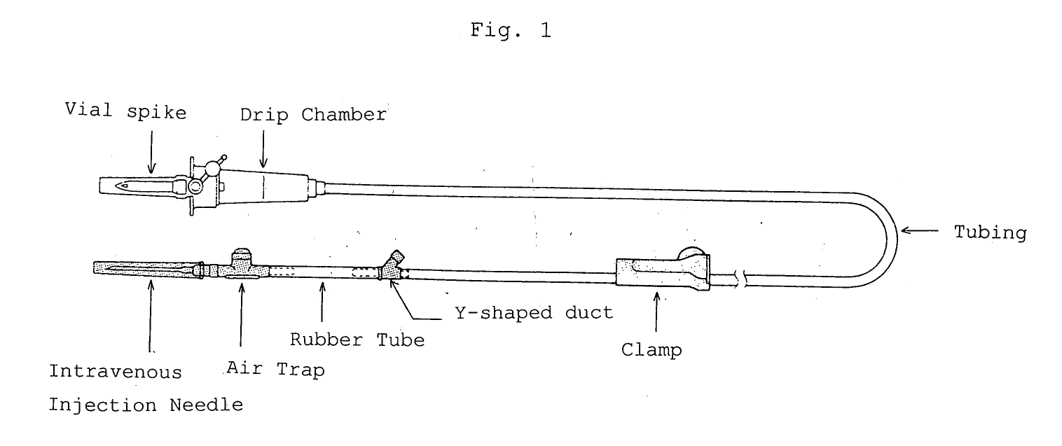

The medical tubing of the present invention can be used as a means for transporting a liquid in medical equipment such as an infusion circuit or a blood circuit, and can forcedly transport a liquid by a pump such as an infusion pump. One example thereof is shown in FIG. 1. The pump presses the tube from the periphery thereof and transports a drug solution to an objective site while squeezing the tubing. As a result, an accurate flow quantity is transported to a body from an infusion bottle or bag. However, in order to withstand squeezing for a long period of time, it is necessary for the tube to have a sufficient elasticity. The medical tubing in the present invention can provide the required elasticity for use in the equipment. The medical equipment is not limited to the above-mentioned infusion circuit and blood circuit, but includes equipment necessary for various liquid transportation in the medical field.

The medical tubing of the present invention is formed from a random copolymer comprising olefin-based monomer units and styrene-based monomer units, and does not contain a plasticizer. Therefore, unlike PVC tube containing a plasticizer, the tubing has a characteristic that a plasticizer does not elute from the tubing. Further, the tubing is suitable for administering drugs because the copolymer does not adsorb oil-soluble drugs such as nitroglycerin. The plasticizer referred to herein means a compound added to conventional thermoplastic polymers such as polyvinyl chloride, and which has a possibility of affecting the human body, such as phthalates, e.g., di(2-ethylhexyl)phthalate, di(isodecyl)phthalate, or adipates, e.g., dioctyl adipate.

The medical tubing of the present invention is obtained by general extrusion molding. If necessary, additives such as antioxidants, lubricants, or the like may be added to the random copolymer. The shape, diameter, length and other parameters of the tube can be selected according to the intended purpose of use, and are not particularly limited.

The obtained tubing is joined with another part such as a drip chamber used in infusion administration, and then packed in a wrapping and sterilized. Ethylene oxide gas sterilization, γ-ray sterilization or electron ray sterilization can be selected as the sterilization method. The medical tubing of the present invention does not contain butadiene units or the like that require additives when conducting γ-ray or electron ray sterilization. It is therefore considered that γ-ray or electron ray sterilization is possible even in the state of not containing additives such as phosphorus-based antioxidants, e.g., bis(2,6-di-t-butyl-4-methylphenyl)pentaerythritol diphosphite or bis(2,4-di-t-butylphenyl)pentaerythritol diphosphite or the like.

### EXAMPLES

The present invention is described below in detail using Examples and Comparative Examples. The invention, however, is not limited to these examples.

In the Examples and Comparative Examples, each test item was measured as follows.

Measurement of Color Tone and Transparency:

After gamma ray irradiation (25 kgy), visually observed.

Eluent Test by Sterilized Infusion Set Standard:

A tube portion of 10 g was cut in about a 1 cm length, and boiled in 100 ml distilled water for 30 minutes. Distilled water was added to accurately make 100 ml, thereby preparing a test liquid. Simultaneously, only distilled water was boiled for 30 minutes to prepare a blank test liquid.

<pH> 20 ml of each of the test liquid and blank test liquid was collected. 1.0 g of potassium chloride was dissolved in water to make 1000 ml, and 1.0 ml thereof was added to each of the test liquid and the blank test liquid. pH change of the liquids was measured by a pH measurement method defined in Japanese Pharmacopoeia. When the pH difference was 2.0 or less, it was considered to be acceptable.

<Heavy metal> 10 ml of a test liquid was collected, and tested according to Method No. 1 of the Heavy Metal Test Method defined in Japanese Pharmacopoeia. For a blank test liquid, 2.0 ml of lead standard liquid was added and tested in the same manner. When color was not dark as compared with the blank test liquid, it was considered to be acceptable.

<Potassium permanganate reducing substance> 10 ml of a test liquid was collected in a stoppered triangular flask, 20.0 ml of 0.002 mol/liter potassium permanganate solution and 1 ml of diluted sulfuric acid were added thereto, and the flask was sealed. The flask was shaken and then allowed to stand for 10 minutes, followed by titration with 0.01 mol/liter sodium thiosulfate solution (5 drops of indicator, starch reagent). Separately, the same procedure was conducted using 10 ml of a blank test liquid. When the difference between a test liquid and the blank test liquid in the amount of 0.002 mol/liter potassium permanganate solution consumed was 2.0 ml or less, it was considered to be acceptable.

<Evaporation residue> 10 ml of a test liquid was evaporated to dryness on a water bath, and the residue was dried at 105° C. for 1 hour. If its weight at that time was 1.0 mg or less, it was considered to be acceptable.

Nitroglycerin Adsorption Property:

60 ml of a nitroglycerin injection (effective component: 50 mg/100 ml, Millisrol injection, manufactured by Nippon Kayaku Co.) was poured into 1 liter of a physiological saline defined in Japanese Pharmacopoeia (manufactured by Otsuka Pharmaceutical Co.), followed by slowly stirring. Sampling was immediately conducted by an injection syringe equipped with an injection needle to prepare a blank sample. A tube of an infusion set was closed with a clamp for controlling flow rate, and a bottle needle of a drip chamber was pierced into the rubber plug of an infusion container. A lower half portion of the drip chamber was filled with the solution by pumping the drip chamber. The clamp for controlling flow rate was gradually released to fill the inside of the tube with the solution, and the tube was attached to an infusion pump FP-2001, manufactured by Nissho Co. Flow rate was set to 36 ml/hr, the clamp for controlling flow rate was opened, the switch was turned on, and then infusion was initiated. Sampling of the solution which flowed out from the end of the tube was chronologically conducted, and the concentration was measured with high performance liquid chromatography. Infusion was conducted for 180 hours, and sampling was conducted every 5 minutes for the first 60 minutes and every 15 minutes after that.

Column: CAPCELL PAK C18 SG120 Å 5 μmφ4.6 mm×250 mm manufactured by Shiseido Co.

Temperature: 30° C.

Mobile phase: methanol:water=11:9

Detector: UV wavelength 210 nm

Flow rate: 0.8 ml/min

If 90% or less of the concentration was observed as compared with the blank concentration, it was considered that there is adsorption.

Pump Flow Rate Stability (250 ml/hr, 24 hrs):

A tube was closed with a clamp for controlling flow rate, and a bottle needle of a drip chamber was pierced into a rubber plug of a container of physiological saline defined in Japanese Pharmacopoeia (manufactured by Otsuka Pharmaceutical Co.) . A lower half portion of the drip chamber was filled with the saline by pumping the drip chamber. The clamp for controlling flow rate was gradually released to fill the inside of the tube with the saline, and the tube was attached to an infusion pump FP-2001, manufactured by Nissho Co. Flow rate was set to 250 ml/hr, the clamp for controlling flow rate was opened, the switch was turned on, and infusion was initiated. The amount of physiological saline which flowed but of the top of the tube was immediately received by a measuring cylinder to measure flow rate per minute (V₀). After 24 hours, flow rate per minute (V₁) was again measured in the same manner. Flow rate change (V₁−V₀/V₀)×100(%) after 24 hours was obtained. It was considered to be acceptable if the change was within ±15%.

Tube State After Finishing Pump Infusion:

After testing the above pump flow rate stability, the surface state, the change in tube diameter and the presence or absence of cracks were observed on the squeezed portion of the tube.

### Example 1

An ethylene-styrene random copolymer (MFR 9 g/10 mins, melting point 71° C.) constituted of 15 mol % styrene and 85 mol % ethylene was extrusion molded at a melting temperature of 200° C. to obtain a tube having an inner diameter of 2.7 mm and an outer diameter of 3.8 mm. Conventional plasticizer was not added.

The obtained tube was cut into a length of about 1.2 m, and passed through a clamp for controlling flow rate. A drip chamber equipped with a vial spike was connected to one end of the tube, and an intravenous needle, an air trap, an isoprene rubber tubing and a Y-shape duct were connected to the other end of the tube to obtain an infusion set as shown in FIG. 1. After sterilization of the tube with γ-ray irradiation, the color tone and transparency of the tube were measured, and an eluted material test according to the sterilized infusion set standard was conducted. And nitroglycerin adsorption property, pump flow rate stability (250 ml/hr, 24 hours) and tube state after finishing of pump infusion were observed. The results obtained are shown in Table 1.

### Example 2

A tube was prepared in the same manner as in Example 1 except that an ethylene-styrene random copolymer (MFR 4 g/10 mins, melting point 79° C.) having a styrene content of 10 mol % and an ethylene content of 90mol % was used in place of the ethylene-styrene random copolymer (MFR 9 g/10 mins, melting point 71° C.) having a styrene content of 15 mol % and ethylene content of 85 mol %, and the same tests were conducted. The results obtained are shown in Table 1.

### Example 3

A tube was prepared in the same manner as in Example 1 except that an ethylene-styrene random copolymer (MFR 5 g/10 mins, melting point 31° C.) having a styrene content of 20 mol % and an ethylene content of 80 mol % was used in place of the ethylene-styrene random copolymer (MFR 9 g/10 mins, melting point 71° C.) having a styrene content of 15 mol % and an ethylene content of 85 mol %, and the same tests were conducted. The results obtained are shown in Table 1.

### Example 4

A tube was prepared in the same manner as in Example 1 except that an ethylene-styrene random copolymer (MFR 0.5 g/10 mins, melting point 68° C.) having a styrene content of 15 mol % and an ethylene content of 85 mol % was used in place of the ethylene-styrene random copolymer (MFR 9 g/10 mins, melting point 71° C.) having a styrene content of 15 mol % and an ethylene content of 85 mol %, and the same tests were conducted. The results obtained are shown in Table 1.

### Comparative Example 1

A tube (inner diameter 2.7 mm, outer diameter 3.8 mm, length 120 cm) obtained from polyvinyl chloride containing 35% by weight of di(2-ethylhexyl)phthalate as a plasticizer was subjected to the same tests as in Example 1. The results obtained are shown in Table 2

### Comparative Example 2

A tube (inner diameter 2.7 mm, outer diameter 3.8 mm, length 120 cm) obtained from syndiotactic 1,2-polybutadiene (manufactured by JSR Corporation, RB810, density 0.901 g/cm³, melting point 71° C., melt index 3 g/10 mins-150° C.-2160 g) was subjected to the same tests as in Example 1. The results obtained are shown in Table 2.

### Comparative Example 3

A tube (inner diameter 2.7 mm, outer diameter 3.8 mm, length 120 cm) obtained from a mixture of 80% by weight of syndiotactic 1,2-polybutadiene (manufactured by JSR Corporation, RB820, density 0.906 g/cm³, melting point 95° C., melt index 3 g/10 mins-150° C.-2160 g) and 20% by weight of styrene-isoprene-styrene block copolymer (manufactured by JSR corporation, SIS5000P, styrene content 15% by weight, density 0.92 g/cm³, MFR 2 g/10 mins-200° C.-5 kg) was subjected to the same tests as in Example 1. The results obtained are shown in Table 2.

### Comparative Example 4

A tube (inner diameter 2.7 mm, outer diameter 3.8 mm, length 120 cm) obtained from a mixed resin of 70% by weight of linear low density polyethylene (manufactured by Dow Chemical Co., PF1140, density 0.895 g/cm³) and 30% by weight of styrene-based elastomer (manufactured by Shell, KRATON G1657, SEBS, styrene content 13% by weight, MFR 8 g/10 mins-200° C.-5.0 kg) was subjected to the same tests as in Example 1. The results obtained are shown in Table 2.

TABLE 1

| | Examples | | | |
|---|---|---|---|---|
| Test | 1 | 2 | 3 | 4 |
| Color tone, | Colorless, | Colorless, | Colorless, | Colorless, |
| transparency | transparent | transparent | transparent | transparent |
| Eluted | Acceptable | Acceptable | Acceptable | Acceptable |
| substance | | | | |
| Nitroglycerin | <5% | <5% | <5% | <5% |
| adsorption | | | | |
| property | | | | |
| (maximum | | | | |
| sorption | | | | |
| ratio) | | | | |
| Pump flow rate | <10% | <10% | <10% | <10% |
| stability | | | | |
| (250 ml/after | | | | |
| 24 hours) | | | | |
| Tube state | No | No | No | No |
| after | abnormality | abnormality | abnormality | abnormality |
| completion of | | | | |
| pump infusion | | | | |
| Total | ∘ | ∘ | ∘ | ∘ |
| evaluation | | | | |

TABLE 2

| | Comparative Examples | | | |
|---|---|---|---|---|
| Test | 1 | 2 | 3 | 4 |
| Color tone, | Colorless, | Yellowed | Yellowed | Colorless, |
| transparency | transparent | | | transparent |
| Eluted | Acceptable | Acceptable | Acceptable | Acceptable |
| substance | | | | |
| Nitroglycerin | 45% | <5% | <5% | <5% |
| adsorption | | | | |
| property | | | | |
| (maximum | | | | |
| sorption ratio) | | | | |
| Pump flow rate | <10% | <10% | <10% | <10% |
| stability | | | | |
| (250 ml/after | | | | |
| 24 hours) | | | | |
| Tube state after | No | Rough | Rough | Rough |
| completion of | abnormality | surface | surface | surface |
| pump infusion | | | | |
| Total | x | x | x | x |
| evaluation | | | | |

The tubes of the above Examples 1-4 were colorless and transparent even after γ-ray sterilization was conducted. The results of eluted substance test by sterilized infusion set standard were acceptable, nitroglycerin adsorption property was a maximum of 5%, pump flow rate variation was within 10%, and no abnormality was observed on the tube surface after finishing pump infusion. On the contrary, in the tube made of polyvinyl chloride containing a plasticizer, nitroglycerin was adsorbed on the tube (Comparative Example 1). The tube comprising syndiotactic 1,2-polybutadine containing no antioxidant (Comparative Example 2) and the tube comprising a mixture of syndiotactic 1,2-polybutadiene and styrene-isoprene-styrene block copolymer (Comparative Example 3) were colored yellow after γ-ray sterilization was conducted. Further, the surface was scraped on squeezing with the pump, and many white powdered materials were generated. In the tube comprising a mixture of linear low density polyethylene and styrene-based elastomer (Comparative Example 4), the surface was also scraped on squeezing with the pump and many white powdered materials were generated.

According to the present invention, a random copolymer of an olefin-based monomer and a styrene-based monomer, not containing chlorine atoms or a plasticizer can provide a medical tubing in which an adsorption of drugs is very small. Further the tubing of the present invention can withstand squeezing action by an infusion pump, has a possibility of undergoing γ-ray sterilization and has a flexibility necessary for clinical use.

What is claimed is:

1. An infusion set for use in an infusion pump for administering an injection solution containing nitroglycerin into an objective site, comprising:
a vial spike to be connected to an infusion bottle or bag containing the injection solution containing nitroglycerin;
tubing connected to the vial spike for conveying the injection solution to the objective site by squeezing of the tube by means of the infusion pump;
a clamp arranged on the tubing for controlling the flow of the injection solution; and
a means for introducing the injection solution into the objective site;
said tubing being a single layer tube formed from a random copolymer consisting of 75-95 mole % of ethylene and 25-5 mole % of a styrene, the MFR (200° C.) of said random copolymer being 0.1-50 g/10 mins, and said tubing not adsorbing nitroglycerin contained in the injection solution.

2. The infusion set as claimed in claim 1, wherein said random copolymer of said tubing is further copolymerized with a monomer containing a glycidyl group, amino group, dimethylamino group, hydroxyl group, carboxyl group, ester group, ether group or isocyanate group, or with a maleic anhydride.

3. The infusion set as claimed in claim 1, wherein said random copolymer of said tubing is a random copolymer consisting of 80-90 mol % ethylene and 20-10 mol % styrene.

4. The infusion set as claimed in claim 1, wherein a content of a block or blocks of two or more continuous units of the ethylene monomer or the styrene monomer in the random copolymer of said tubing is 10% or less.

5. The infusion set as claimed in claim 1, wherein the tubing is sterilized with a γ-ray or an electron ray.

6. The infusion set as claimed in claim 1, wherein the MFR (200° C.) of said random copolymer of said tubing is 0.3-10 g/10 mins.

7. A method for administering an injection solution containing nitroglycerin to a patient, comprising:
providing an infusion bottle or bag containing the injection solution containing nitroglycerin;
connecting said infusion bottle or bag containing the injection solution to tubing for conveying the injection solution to a patient;
connecting said tubing to a means for introducing the injection solution into an objective site in the patient;
inserting said means into the objective site; and
repeatedly squeezing said tubing with an infusion pump to forcibly convey said injection solution to the patient;
said tubing being a single layer tube formed from a random copolymer consisting of 75-95 mole % of ethylene and 25-5 mole % of a styrene, the MFR (200° C.) of said random copolymer being 0.1-50 g/10 mins, and said tubing not adsorbing nitroglycerin contained in the injection solution.

8. The method of claim 7, wherein said random copolymer of said tubing is further copolymerized with a monomer containing a glycidyl group, amino group, dimethylamino group, hydroxyl group, carboxyl group, ester group, ether group or isocyanate group, or with a maleic anhydride.

9. The method of claim 7, wherein said random copolymer of said tubing is a random copolymer consisting of 80-90 mol % ethylene and 20-10 mol % styrene.

10. The method of claim 7, wherein a content of a block or blocks of two or more continuous units of the ethylene monomer or the styrene monomer in the random copolymer of said tubing is 10% or less.

11. The method of claim 7, wherein the tubing is sterilized with a γ-ray or an electron ray.

12. The method of claim 7, wherein the MFR (200° C.) of said random copolymer of said tubing is 0.3-10 g/10 mins.
